(19) United States
(12) Patent Application Publication
(10) Pub. No.: US 2011/0237965 A1
(43) Pub. Date: Sep. 29, 2011

(54) PULSE WAVE DETECTOR

(75) Inventors: Takuya Hayashi, Kyoto (JP); Katsumi Imada, Nara (JP); Susumu Fukushima, Osaka (JP)

(73) Assignee: Panasonic Corporation, Osaka (JP)

(21) Appl. No.: 13/047,243

(22) Filed: Mar. 14, 2011

(30) Foreign Application Priority Data
Mar. 24, 2010 (JP) 2010-067596

(51) Int. Cl. A61B 5/02

(52) U.S. Cl. 600/500

(57) ABSTRACT

A pulse wave detector includes a) a light source repeatedly turned on and off, b) a light receiving element for receiving light, and c) an arithmetic processor for processing an output value acquired through the light receiving element. The arithmetic processor performs arithmetic processing for calculating the difference between a first output value acquired through the light receiving element when the light source is turned on and a second output value acquired through the light receiving element when the light source is turned off. With this structure, a pulse wave detector capable of detecting pulse waves even under the conditions where external light intensity varies can be provided.

### BACKGROUND OF THE INVENTION

1. Field of the Invention

The present invention relates to a pulse wave detector for detecting human pulse waves, which are obtained by arithmetically processing signals in a characteristic manner.

2. Background Art

FIG. 8 is a diagram showing a conventional pulse wave detector. With reference to FIG. 8, conventional pulse wave detector 100 has sensor 100A, and driver 170 for driving the sensor and processing signals. Sensor 100A includes light source 20 and light receiving element 21.

The principle of pulse wave detection is as follows. Light incident from light source 20 on part of a body is absorbed and reflected by oxygen or reduced hemoglobin in the blood flowing through the blood vessels of the body. By detecting the intensity of the light reflected (reflected light), pulse waves indicating the flow of the blood can be detected. The intensity of the light detected in light receiving element 21 is converted into an electrical signal, and the electrical signal is processed in signal processor 22 and output in a form suitable for the intended purpose.

Major methods for lighting light source 20 include a DC lighting method and a pulse lighting method. In the case of the DC lighting method, driver 170 can be formed of a simple structure, but it is difficult to discriminate between DC light from the light source and external light. For example, under intense external light, it is difficult to extract pulse waves from the light detected in light receiving element 21.

FIG. 9 is a diagram showing a conventional pulse wave detector using a pulse lighting method. With reference to FIG. 9, conventional pulse wave detector 101 has sensor 101A, and driver 171 for driving the sensor and processing signals. In the pulse lighting method, driver 171 requires pulse signal generator 23.

FIG. 10A is a waveform chart of pulse signals in the conventional pulse wave detector using the pulse lighting method. With reference to FIG. 10A, pulse signals generated by conventional pulse signal generator 23 drive light source 24, and light in a pulse form is incident from light source 24 on part of a body (fingertip). Light receiving element 25 detects the reflected light from the fingertip (including pulse wave signals modulated by pulse signals).

FIG. 10B is a waveform chart of signals acquired through the light receiving element of the conventional pulse wave detector using the pulse lighting method. With reference to FIG. 10B, an electrical signal detected through the light receiving element of the conventional pulse wave detector is input to high-pass filter 26. High-pass filter 26 attenuates the DC component mainly included in external light.

The pulse signal generated by pulse signal generator 23 and the output signal from high-pass filter 26 are input to lock-in amplifier 27, and the output signal from high-pass filter 26 is demodulated by the pulse signal.

FIG. 11A is a waveform chart of output signals from the conventional pulse wave detector using the pulse wave lighting method. With reference to FIG. 11A, the electrical signal output from lock-in amplifier 27 (the signal including a pulse wave signal) is passed through amplifier 28 and filter 29, and moreover the polarity of the processed signal is inverted. Thereby, even under intense external light, a pulse wave signal whose component of the external light is removed can be detected. The above technique is disclosed in Japanese Patent Unexamined Publication No. 2005-160641.

Conventional pulse wave detector 101 is capable of detecting pulse waves (see the waveform of FIG. 11A) even under intense external light provided the external light intensity is steady. However, for instance, suppose the detector is mounted on the steering wheel, for example, above the driver seat of a vehicle, and the vehicle moves from a sunny place to the shade while running. Under such conditions where external light intensity varies with time, (especially when the intensity of external light that cannot be attenuated by high-pass filter 26 or filter 29 varies with time), the pulse waves cannot be detected.

FIG. 11B is a waveform chart of output signals from the conventional pulse wave detector using the pulse lighting method when the external light intensity varies with time. With reference to FIG. 11B, in the waveform of the output signals (pulse wave signals) from filter 29 under the conditions where the external light intensity varies with time, the pulse wave signals change as shown in waveform parts 30 as the external light intensity varies with time.

### SUMMARY OF THE INVENTION

A pulse wave detector of the present invention is capable of detecting pulse waves even under the conditions where external light intensity varies.

The pulse wave detector of the present invention includes the following elements:
a) a light source repeatedly turned on and off;
b) a light receiving element for receiving light; and
c) an arithmetic processor for processing an output value acquired through the light receiving element.
   The arithmetic processor performs arithmetic processing for calculating the difference between a first output value acquired through the light receiving element when the light source is turned on and a second output value acquired through the light receiving element when the light source is turned off.

The pulse wave detector of the present invention has a light source repeatedly turned on and off. The light receiving element receives the reflected light from part of a living body when the light source is turned on. The output signal from the light receiving element at this time has the first output value. The first output value includes external light noise and biological information. In contrast, the output value from the light receiving element when the light source is turned off has the second output value. The second output value includes the external light noise. Thus, the arithmetic processing for calculating the difference between the first output value and the second output value in the arithmetic processor cancels the component of the external light noise in the first output value. Therefore, an accurate pulse signal can be detected.

### BRIEF DESCRIPTION OF DRAWINGS

FIG. 1 is a block diagram of a pulse wave detector in accordance with an exemplary embodiment of the present invention.

FIG. 2 shows a waveform chart of signals acquired through a light receiving element of the pulse wave detector in accordance with the exemplary embodiment when external light intensity is constant.

FIG. 3A is a waveform chart of signals obtained by arithmetic processing output of the pulse wave detector in accordance with the exemplary embodiment.

FIG. 3B is a waveform chart of output signals from the pulse wave detector in accordance with the exemplary embodiment.

FIG. 3C is an inverted waveform chart of the output signals shown in FIG. 3B.

FIG. 4 shows a waveform chart of signals acquired through the light receiving element of the pulse wave detector in accordance with the exemplary embodiment when the external light intensity varies.

FIG. 5A is a waveform chart of signals obtained by the arithmetic processing output of the pulse wave detector in accordance with the exemplary embodiment when the external light intensity varies.

FIG. 5B is a waveform chart of output signals from the pulse wave detector in accordance with the exemplary embodiment when the external light intensity varies.

FIG. 5C is an inverted waveform chart of the output signals shown in FIG. 5B.

FIG. 6 is an explanatory view of acquisition of first output values and second output values.

FIG. 7 shows a signal waveform chart of the first output values and the second output values acquired by a sensor shown in FIG. 6.

FIG. 8 is a diagram showing a conventional pulse wave detector.

FIG. 9 is a diagram showing a conventional pulse wave detector using a pulse lighting method.

FIG. 10A is a waveform chart of pulse signals in the conventional pulse wave detector using the pulse lighting method.

FIG. 10B is a waveform chart of signals acquired through a light receiving element of the conventional pulse wave detector using the pulse lighting method.

FIG. 11A is a waveform chart of output signals from the conventional pulse wave detector using the pulse wave lighting method.

FIG. 11B is a waveform chart of output signals from the conventional pulse wave detector using the pulse lighting method when the external light intensity varies.

### DETAILED DESCRIPTION OF THE INVENTION

### Exemplary Embodiment

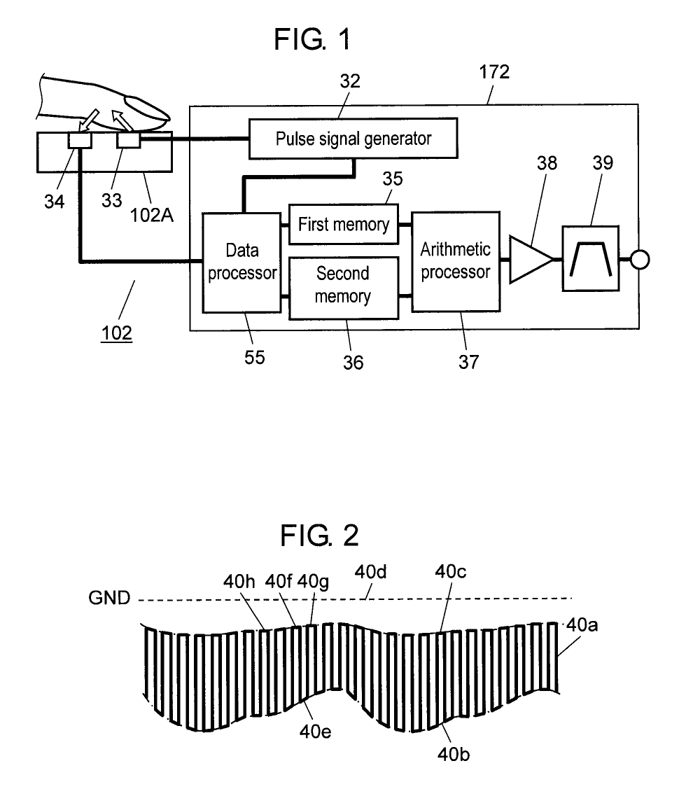

Hereinafter, a description is provided for a pulse wave detector in accordance with the exemplary embodiment of the present invention with reference to FIG. 1.

FIG. 1 is a block diagram of the pulse wave detector in accordance with the exemplary embodiment of the present invention. With reference to FIG. 1, pulse wave detector 102 has sensor 102A, and driver 172 for driving the sensor and processing signals. Sensor 102A includes light source 33 and light receiving element 34. Driver 172 includes data processor 55 electrically connected to light receiving element 34, first memory 35 electrically connected to data processor 55, second memory 36 also electrically connected to data processor 55, and pulse signal generator 32 for supplying pulse signals to light source 33 and to data processor 55. Driver 172 further includes arithmetic processor 37 electrically connected to first memory 35 and second memory 36, amplifier 38 electrically connected to arithmetic processor 37, and filter 39 electrically connected to amplifier 38.

Because light source 33 is driven based on the pulse signals output from pulse signal generator 32, the light radiated from the light source is repeatedly turned on and off in response to the waveform of the pulse signals.

Hereinafter, as an example, a description is provided on the assumption that a finger is placed on the top surfaces of light source 33 and light receiving element 34.

When the light source is turned on, part of the light radiated from the light source is incident on the finger, and the light absorbed and reflected by oxygen or reduced hemoglobin in the blood flowing through the blood vessels of the finger is received by light receiving element 34. The intensity of the received light is converted into an electrical signal, and this light reception signal is output to data processor 55 in driver 172.

On the basis of the pulse signals supplied from pulse signal generator 32, data processor 55 sorts the light reception signals into those having first output values and those having second output values. That is, the first output values output from data processor 55 are input to first memory 35. Similarly, the second output values output from data processor 55 are input to second memory 36. Here, the first output value indicates an output signal acquired through light receiving element 34 when light source 33 is turned on. The second output value indicates an output signal acquired through light receiving element 34 when light source 33 is turned off.

Depending on the state of the pulse signal supplied from pulse signal generator 32, data processor 55 determines whether light source 33 is turned on or off. Further, the data processor determines whether the light reception signal acquired from light receiving element 34 has the first output value or the second output value. At this time, if there is a time lag between the pulse signal input from pulse signal generator 32 and the light reception signal input from light receiving element 34, data processor 55 may sort the light reception signals into those having the first output values and those having the second output values, allowing for the time lag. This allows the light reception signals to be accurately sorted into those having the first output values and those having the second output values.

The first output value input to first memory 35 is stored in first memory 35 for a fixed period of time. Similarly, the second output value input to second memory 36 is stored in second memory 36 for a fixed period of time.

The first output value, i.e. the output value from light receiving element 34 when light source 33 is turned on, includes external light noise and a pulse wave signal acquired from the reflected light from the blood vessels of the finger. In contrast, the second output value, i.e. the output value from the light receiving element when light source 33 is turned off, mainly includes the external light noise.

Thus, arithmetic processing for calculating the difference between the first output value from first memory 35 and the second output value from second memory 36 in arithmetic processor 37 cancels the component of the external light noise in the first output value. Therefore, an accurate pulse wave signal can be detected.

Arithmetic processor 37 outputs the detected pulse wave signal to amplifier 38, and the pulse wave signal is amplified in amplifier 38. The amplified pulse wave signal is input to filter 39, which suppresses the DC component and high-frequency noise.

Next, the operation of pulse wave detector 102 of the present invention is described with reference to FIG. 2 through FIG. 5.

FIG. 2 shows a waveform chart of signals acquired through the light receiving element of the pulse wave detector in accordance with the exemplary embodiment of the present invention when external light intensity is constant. In FIG. 2, the vertical axis shows an amplitude value of the signal (in the lower position along the vertical axis, the amplitude value being the larger), and the horizontal axis shows a time (in the more right position along the horizontal axis, the time being the more advanced).

Signal waveform 40a indicates signals output from light receiving element 34 when the external light intensity is substantially constant. Signal waveform 40a is a rectangular waveform since light source 33 is driven by pulse signals output from pulse signal generator 32.

Broken line waveform 40b indicates pulse wave signals. The portions in contact with waveform 40b in signal waveform 40a substantially correspond to the first output values. Further, broken line waveform 40c mainly indicates external light noise. The portions in contact with waveform 40c in signal waveform 40a substantially correspond to the second output values. That is, in the portions in contact with waveform 40b in signal waveform 40a, light source 33 is turned on. In the portions in contact with waveform 40c, light source 33 is turned off. Broken straight line 40d indicates the ground level. The potential difference between waveform 40d and waveform 40c shows a voltage output from light receiving element 34, which is produced by the reception of external light noise by light receiving element 34.

In spite of the condition where the external light intensity is substantially constant, waveform 40c indicating external light noise does not take a constant value. This is because part of the external light is transmitted through the finger and reaches light receiving element 34. That is, a pulse wave is superimposed on the external light when the external light is transmitted through the finger. Further, changes in the position or pressing pressure of the finger placed on the top surface of light receiving element 34 greatly vary both of the intensity of the external light directly reaching light receiving element 34 and the intensity of the external light transmitted through the finger and reaching light receiving element 34. This is also one of the factors in the inconstant values.

As described above, part of the pulse wave component is superimposed on the external light noise. In the pulse wave detector of the exemplary embodiment, the pulse wave superimposed on the external light noise is handled as part of noise.

Data processor 55 for receiving signal waveform 40a determines which signal of those having the first output value and the second output value is being input to data processor 55, based on the pulse signal supplied from pulse signal generator 32.

For instance, specifically, data processor 55 determines that waveform portion 40f corresponds to the (n−1)-th second output value, based on the pulse signal supplied from pulse signal generator 32, and outputs the data on waveform portion 40f to second memory 36. Next, the data processor determines that waveform portion 40e corresponds to the n-th first output value, and outputs the data on waveform portion 40e to first memory 35. Further, the data processor determines that waveform portion 40g corresponds to the n-th second output value, and outputs the data on waveform portion 40g to second memory 36.

Arithmetic processor 37 calls and acquires the n-th first output value stored in first memory 35. The arithmetic processor calls and acquires the n-th second output value (or the (n−1)-th second output value) stored in second memory 36. Then, the arithmetic processor performs arithmetic processing for calculating the difference between the n-th first output value and the n-th second output value (or the (n−1)-th second output value).

Here, the magnitude of the external light noise included in the n-th first output value is substantially equal to the magnitude of the external light noise included in the n-th second output value (or the (n−1)-th second output value). Thus, the component of the external light noise of the n-th first output value is substantially cancelled, and a pulse wave signal can be detected accurately.

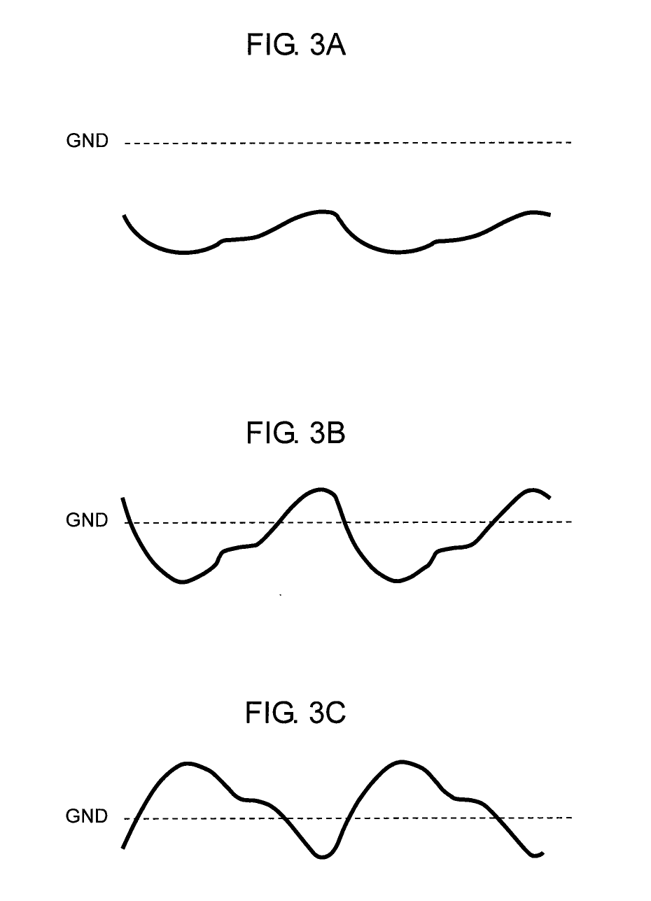

FIG. 3A is a waveform chart of signals obtained by arithmetic processing output of the pulse wave detector in accordance with the exemplary embodiment of the present invention. FIG. 3B is a waveform chart of output signals from the pulse wave detector in accordance with the exemplary embodiment. FIG. 3C is an inverted waveform chart of the output signals shown in FIG. 3B.

With reference to FIGS. 3A, 3B, and 3C, the pulse wave signals output from arithmetic processor 37 have a waveform of FIG. 3A, for example. Next, the signals passed through amplifier 38 and filter 39 have a waveform of FIG. 3B. Further, when the waveform of FIG. 3B is inverted, the waveform of FIG. 3C is obtained.

The first output value may be a plurality of amplitude values of signal waveform 40a in the period during which light source 33 is turned on. The first output value may be an average of the amplitude values of signal waveform 40a in the period during which light source 33 is turned on. The first output value may be an amplitude value acquired at the timing at the center of the period during which light source 33 is turned on. In short, an amplitude value of signal waveform 40a in the period during which light source 33 is turned on can be used.

In the above description, arithmetic processor 37 performs arithmetic processing for calculating the difference, using the (n−1)-th second output value (the output value corresponding to waveform portion 40f) or the n-th second output value (the output value corresponding to waveform portion 40g), which is acquired at a timing closest to the timing when the n-th first output value (the output value corresponding to waveform portion 40e) is acquired. This is because the use of the (n−1)-th second output value or the n-th second output value, which is acquired at a timing closest to the timing when the n-th first output value is acquired, allows the magnitudes of the external light noise component in the respective output values to approximate to each other. However, the present invention is not limited to this method. For instance, arithmetic processor 37 may perform arithmetic processing for calculating the difference, using the (n−3)-th second output value corresponding to waveform portion 40h (the waveform portion not adjacent to waveform portion 40e) of FIG. 2. This is because in the case where the external light noise varies little with time as shown in FIG. 2, the second output values do not need to be acquired at sampling intervals equal to those of the first output values. That is, this is because the difference between the amplitude value of waveform portion 40f and the amplitude value of waveform portion 40h is small. This method can reduce the power consumption of data processor 55 and second memory 36, for example.

In order to implement a structure where the sampling interval of the second output values is different from the sampling interval of the first output values, the magnitude of temporal variations in external light noise needs to be obtained. For this purpose, the magnitude of temporal variations in external light noise may be obtained by analyzing temporal variations in the second output values stored in second memory 36.

Specifically, for instance, arithmetic processor 37 controls the operation of data processor 55 and second memory 36 in response to the obtained magnitude of temporal variations in external light noise and changes the sampling intervals at which the second output values are acquired. That is, in the case where the temporal variations in external light noise are small, the sampling intervals at which the second output values are acquired are increased. In the case where the temporal variations in external light noise are large, the sampling intervals at which the second output values are acquired are reduced. Such a structure can reduce the power consumption while enhancing the accuracy of the pulse wave signals.

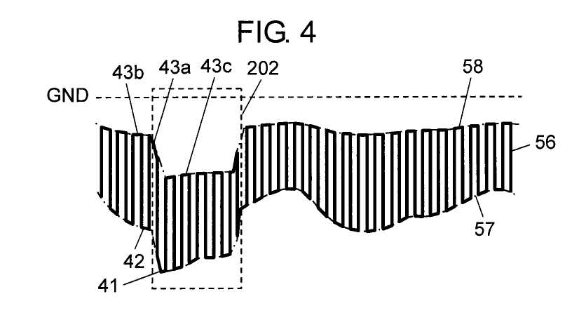

FIG. 4 shows a waveform chart of signals acquired through the light receiving element of the pulse wave detector in accordance with the exemplary embodiment of the present invention when the external light intensity varies. In FIG. 4, output signal waveform 56 acquired through light receiving element 34 when the external noise varies greatly in a short period of time shows a sudden increase in the external light intensity in section 202. This is also understood from temporal variations in the amplitude value of waveform 58 (shown by a broken line) that indicate changes mainly in the external light noise.

Waveform portion 41 substantially in contact with waveform 57 corresponds to the n-th first output value; waveform portion 42 corresponds to the (n−1)-th first output value. Waveform portion 43a substantially in contact with waveform 58 corresponds to the (n−1)-th second output value; waveform portion 43b corresponds to the (n−2)-th second output value. Waveform portion 43c corresponds to the (n+1)-th second output value.

In order to remove the external light noise included in the n-th first output value (the output value corresponding to waveform portion 41), arithmetic processor 37 calculates the difference between the n-th first output value and the (n−1)-th second output value (the output value corresponding to waveform portion 43a). This allows an accurate pulse wave signal to be obtained even when the external light noise suddenly changes in a short period of time.

In the above description, arithmetic processing is performed so as to calculate the difference between the n-th first output value and the (n−1)-th second output value. However, arithmetic processing may be performed so as to calculate the difference between the n-th first output value and the (n+1)-th second output value (the output value corresponding to waveform portion 43c, which is not temporally adjacent to waveform portion 41). This is because the amplitude value of the external light noise varies little with time in section 202. This eliminates the need for storing all the acquired second output values in second memory 36. Thus, the memory size of second memory 36 and the power consumption can be reduced.

Arithmetic processor 37 may determine the ordinal number of the second output value from which the difference of the first output value is calculated, based on the temporal variations in the second output value. However, it is not advisable to use the (n−2)-th second output value corresponding to waveform portion 43b for calculating the difference from the n-th first output value when the external light noise of the n-th first output value is removed. This is because, as shown by signal waveform 58, the amplitude value of the external light noise varies greatly with time. In such a case, arithmetic processor 37 of the pulse wave detector of the present invention performs the following arithmetic processing so that a pulse wave signal having fewer errors is detected.

That is, arithmetic processor 37 calculates a first average value, i.e. an average value of the n-th first output value (the output value corresponding to waveform portion 41) and the (n−1)-th first output value (the output value corresponding to waveform portion 42). Then, the arithmetic processor performs arithmetic processing for calculating the difference between this first average value and a second output value. This processing can suppress errors in a pulse wave signal to be detected when the amplitude value of the external light noise varies greatly with time.

In the above description, arithmetic processor 37 may perform arithmetic processing for calculating the difference between the first average value and the (n−1)-th second output value (the output value corresponding to waveform portion 43a). The timing when the (n−1)-th second output value is acquired is between the timing when the n-th first output value is acquired and the timing when the (n−1)-th first output value is acquired. Thus, this processing can enhance the accuracy of the pulse wave signal to be detected.

Similarly, arithmetic processor 37 may derive a pulse wave signal in the following manner. The arithmetic processor calculates a second average value, i.e. an average value of the (n−2)-th second output value (the output value corresponding to waveform portion 43b) and the (n−1)-th second output value (the output value corresponding to waveform portion 43a). Then, the arithmetic processor performs arithmetic processing for calculating the difference between a first output value and the second average value. This processing can suppress errors in a pulse wave signal to be detected when the amplitude value of the external light noise varies greatly with time.

In the above description, arithmetic processor 37 may perform arithmetic processing for calculating the difference between the (n−1)-th first output value (the output value corresponding to waveform portion 42) and the second average value. The timing when the (n−1)-th first output value is acquired is between the timing when the (n−2)-th second output value is acquired and the timing when the (n−1)-th second output value is acquired. Thus, this processing can enhance the accuracy of the pulse wave signal to be detected.

Whether arithmetic processor 37 averages the first output values or the second output values may be determined on the basis of the magnitude of the temporal variations in the second output value. That is, only in the case of great temporal variations in the amplitude value of external light noise, arithmetic processing for calculating the average value of the first output values or the second output values is performed so that errors in the pulse wave signal to be derived are suppressed. In the other cases, the arithmetic processing for calculating the average value of the first output values or the second output values is not performed so that the power consumption is reduced.

Alternatively, on the basis of the magnitude of the temporal variations in the second output value, the frequency of the pulse signals generated in pulse signal generator 32 may be changed. This structure flexibly reduces errors in a pulse wave signal even in an environment with great temporal variations in the amplitude value of external light noise.

The first output values and second output values acquired may be stored in first memory 35 or second memory 36, together with the information on the time of acquisition and the order of acquisition. This allows arithmetic processor 37 to perform the above arithmetic processing easily.

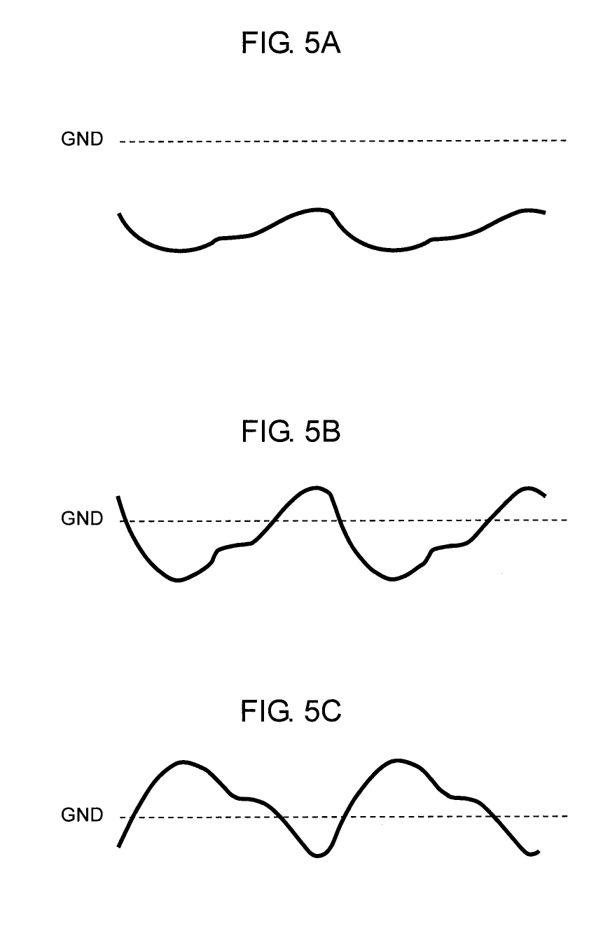

FIG. 5A is a waveform chart of signals obtained by the arithmetic processing output of the pulse wave detector in accordance with the exemplary embodiment of the present invention when the external light intensity varies. FIG. 5B is a waveform chart of output signals from the pulse wave detector in accordance with the exemplary embodiment when the external light intensity varies. FIG. 5C is an inverted waveform chart of the output signals shown in FIG. 5B.

FIGS. 5A, 5B, and 5C correspond to FIGS. 3A, 3B, and 3C, respectively, and the signal waveforms are substantially identical. That is, even when the external light intensity suddenly changes, the pulse wave signals output from arithmetic processor 37 have a waveform of FIG. 5A. Next, the signals passed through amplifier 38 and filter 39 have a waveform of FIG. 5B. Further, when the waveform of FIG. 5B is inverted, the waveform of FIG. 5C is obtained.

With reference to FIG. 1, a description is provided for a structure where arithmetic processor 37, amplifier 38, and filter 39 are disposed separately. However, the present invention is not limited to this structure. A structure where arithmetic processor 37 also functions as amplifier 38 and filter 39 and amplifier 38 and filter 39 are eliminated may be used. With this structure, a small pulse wave detector can be provided.

The above description shows a case where the light from light source 33 is radiated to a fingertip as an example. However, the present invention is not limited to this case. The light may be radiated to any site of a body where pulse waves are observed.

FIG. 1 shows pulse wave detector 102 that includes first memory 35 and second memory 36. However, the pulse wave detector may be implemented so as to include driver 172 that has only either one of first memory 35 and memory 36. For example, driver 172 includes only second memory 36 for recording only second output values. Then, the difference is calculated between a first output value directly input to arithmetic processor 37 and a second output value recorded in second memory 36. With such a structure, a small, inexpensive pulse wave detector can be provided.

Next, hereinafter, a description is provided for the reason why the pulse wave detector of the present invention uses a pulse lighting method.

One of the features of the pulse wave detector of the present invention is to calculate the difference between a first output value and a second output value that includes external light noise.

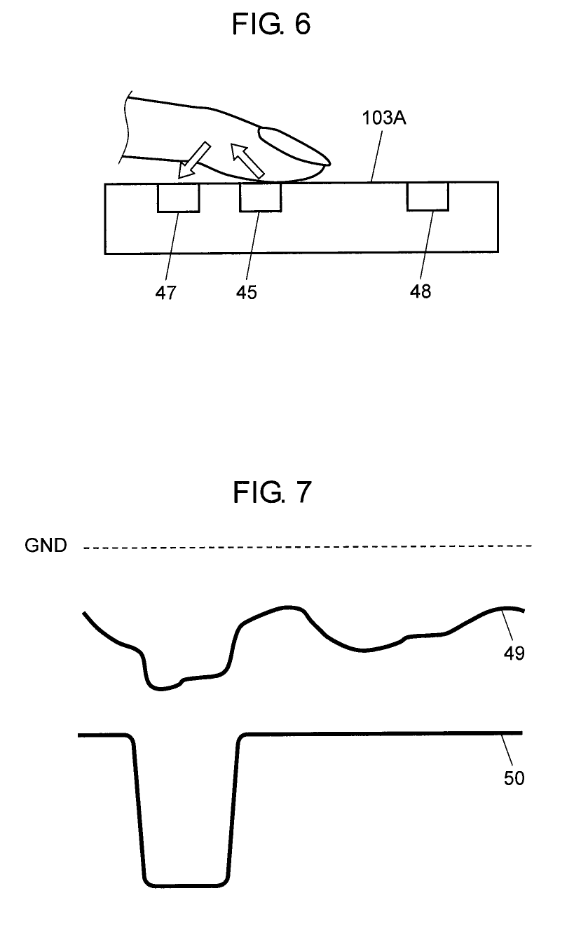

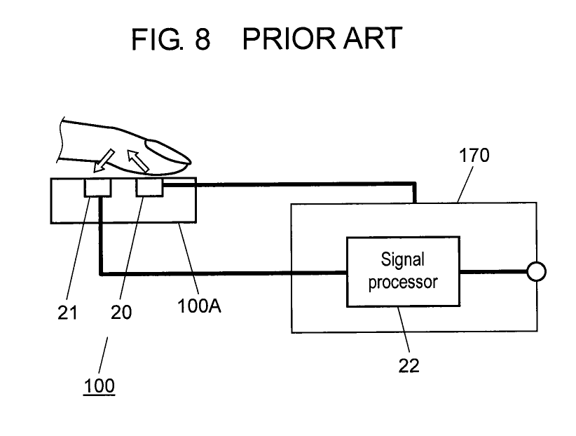

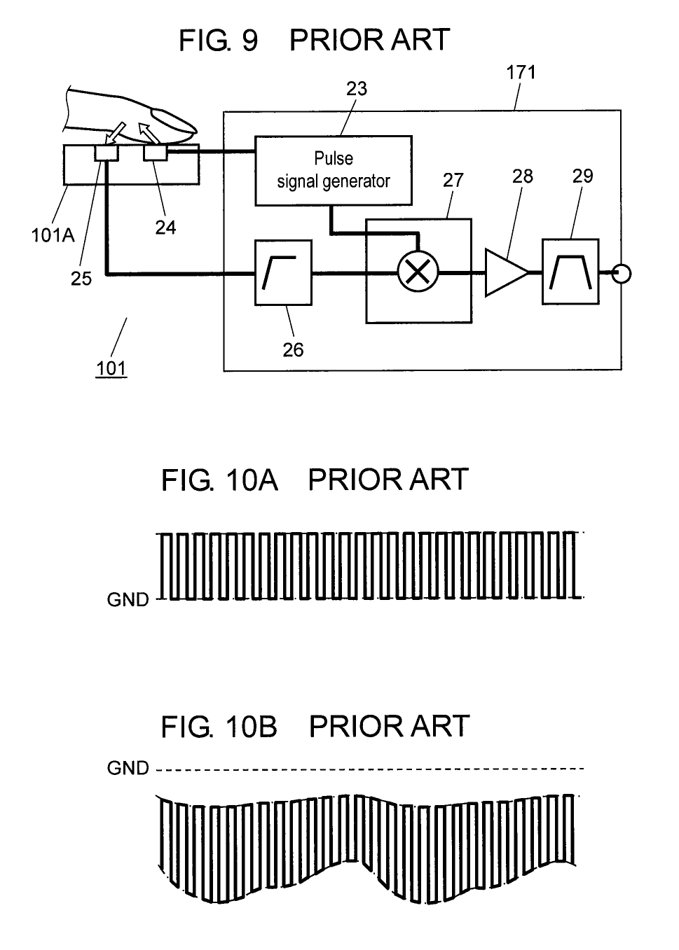

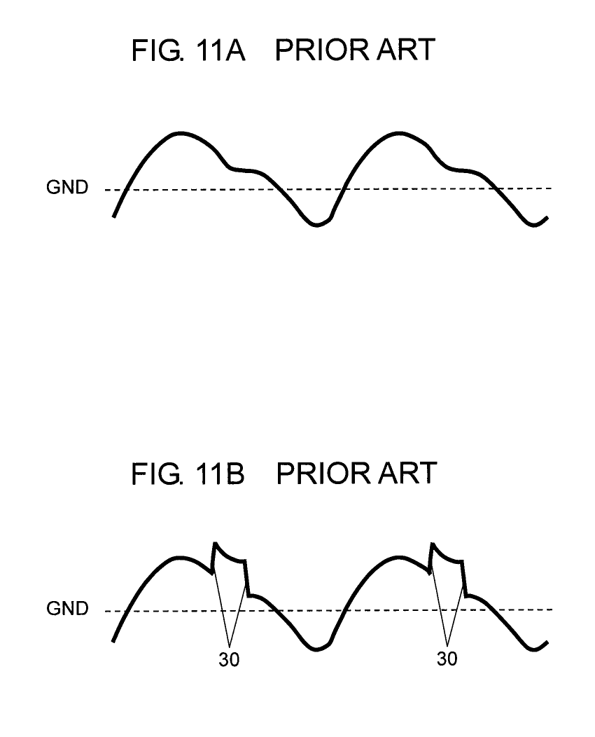

FIG. 6 is an explanatory view of acquisition of first output values and second output values. With reference to FIG. 6, sensor 103A includes light source 45, light receiving element 47 for receiving the reflected light from a finger and the external light, and light receiving element 48 whose top surface is not covered with part of a living body, such as the finger.

Light source 45 is normally turned on by a driving signal. Part of the light radiated from light source 45 is incident on the finger (part of the body), and the light absorbed and reflected by oxygen or reduced hemoglobin in the blood flowing through the blood vessels of the finger tip is detected in light receiving element 47.

FIG. 7 shows a signal waveform chart of the first output values and the second output values acquired by the sensor shown in FIG. 6. With reference to FIG. 7, signal waveform 49 corresponds to the first output values detected in light receiving element 47. Signal waveform 50 corresponds to the second output values detected in light receiving element 48.

Unlike the waveforms of FIG. 2 and FIG. 4, the signals detected in light receiving element 47 are not modulated by pulse signals and are continuous signals. Light receiving element 48 mainly receives external light. The output signals from light receiving element 48 are also continuous signals.

Since light receiving element 48 for detecting only external light noise can continuously acquire external light noise as shown by sensor 103A of FIG. 6, it seems that a pulse wave signal can be obtained accurately. However, the external light noise included in the output signal from light receiving element 47 is attenuated by the influence of the finger placed above light receiving element 47. Thus, the external light noise received in light receiving element 47 greatly differs from the external light noise received in light receiving element 48 in the amplitude value. Here, as a precondition to calculating the difference between the first output value (the signal detected in light receiving element 47) and the second output value (the output signal from light receiving element 48), the second output value needs to be multiplied by a factor of the attenuation of the external light noise caused by a finger, for example.

However, because the factor of the attenuation of the external light noise caused by the finger, for example, greatly varies with the position, pressing pressure or the like of the finger on the top surface of sensor 103A, it is difficult to obtain the value. For this reason, in the method for continuously detecting first output values and second output values with sensor 103A of FIG. 6, it is difficult to detect pulse wave signals accurately.

For the above reason, pulse wave detector 102 of the present invention uses a pulse lighting method. In the pulse lighting method, errors in a pulse wave signal can be suppressed even when the reception level of external light is varied by the finger, for example.

As described above, the pulse wave detector of the present invention is capable of detecting pulse waves even under the conditions where the external light intensity varies. Thus, even under the conditions where the external light intensity varies, e.g. in the case where the detector is mounted on the steering wheel above the driver seat of a vehicle and the vehicle moves from a sunny place to the shade while running, a pulse wave can be detected.

What is claimed is:

1. A pulse wave detector comprising:
a) a light source repeatedly turned on and off;
b) a light receiving element for receiving light; and
c) an arithmetic processor for processing an output value acquired through the light receiving element,
wherein the arithmetic processor performs arithmetic processing for calculating a difference between a first output value acquired through the light receiving element when the light source is turned on and a second output value acquired through the light receiving element when the light source is turned off.

2. The pulse wave detector of claim 1, wherein
the arithmetic processor performs arithmetic processing for calculating an average of an n-th first output value acquired when the light source is tuned on at an n-th time and an (n−1)-th first output value acquired when the light source is turned on at an (n−1)-th time, as a first average value, and
the arithmetic processor uses the average value as the first output value.

3. The pulse wave detector of claim 1, wherein
the arithmetic processor performs arithmetic processing for calculating an average of an n-th second output value acquired when the light source is tuned off at an n-th time and an (n−1)-th second output value acquired when the light source is turned off at an (n−1)-th time, as a second average value, and
the arithmetic processor uses the average value as the second output value.

4. The pulse wave detector of claim 2, wherein
the light receiving element acquires an (n−1)-th second output value in a period between a period during which the n-th first output value is acquired and a period during which the (n−1)-th first output value is acquired, and
the arithmetic processor uses the (n−1)-th second output value as the second output value.

5. The pulse wave detector of claim 3, wherein
the light receiving element acquires an n-th first output value in a period between a period during which the n-th second output value is acquired and a period during which the (n−1)-th second output value is acquired, and
the arithmetic processor uses the n-th first output value as the first output value.

6. The pulse wave detector of claim 1, further comprising a memory, wherein
at least one of the first output value and the second output value is stored in the memory, and
the arithmetic processor performs arithmetic processing using at least one of the first output value and the second output value stored in the memory.
